(19) United States
(12) Patent Application Publication
(10) Pub. No.: US 2013/0158327 A1
(43) Pub. Date: Jun. 20, 2013

(54) HYDROCARBON DEHYDROGENATION WITH INERT DILUENT

(71) Applicants: Laura E. Leonard, Western Springs, IL (US); Andrea G. Bozzano, Northbrook, IL (US); Gavin P. Towler, Inverness, IL (US)

(72) Inventors: Laura E. Leonard, Western Springs, IL (US); Andrea G. Bozzano, Northbrook, IL (US); Gavin P. Towler, Inverness, IL (US)

(73) Assignee: UOP LLC, Des Plaines, IL (US)

(21) Appl. No.: 13/328,931

(22) Filed: Dec. 16, 2011

(51) Int. Cl. C07C 5/333

(52) U.S. Cl. 585/655; 585/659; 585/654

(57) ABSTRACT

A hydrocarbon dehydrogenation process includes providing the hydrocarbon feed to a reactor. The hydrocarbon feed includes at least one hydrocarbon selected from light paraffins, heavy paraffins, or combinations thereof. The process further includes introducing an inert diluent into the feed stream, contacting the feed stream and the inert diluent with a catalyst in the reactor, and flowing an effluent stream out of the reactor.

### FIELD OF THE INVENTION

The invention relates generally to hydrocarbon dehydrogenation processes, and more particularly to a hydrocarbon dehydrogenation process using an inert diluent.

### BACKGROUND OF THE INVENTION

Dehydrogenation is a reversible, endothermic reaction with complicated thermodynamic constraints. The equilibrium conversion increases at higher temperatures in the reactor, as expected. However, increasing the reactor temperature is not a practical option because undesirable side reactions, coke formation, and catalyst deactivation are also increased. Lower partial pressure of the reaction products (e.g., hydrogen and mono-olefin) also increases the dehydrogenation conversion rate. However, simply decreasing the reactor pressure also has downsides such as increased equipment size and cost, increased utility consumption, and in some cases operating at least a portion of the reactor section or separation section under vacuum. In the case of light paraffin dehydrogenation substantially decreasing the reactor pressure would require operating the reactor effluent compressor suction under vacuum, which is undesirable. Dehydrogenation of heavier paraffins is also practiced, for example for the production of detergent range olefins and alkylates. While dehydrogenation of heavier paraffins typically is not practiced under vacuum, lower pressure operation in this case does suffer from disadvantages of larger equipment size and utilities.

Known catalytic light paraffin dehydrogenation processes include, for example, the Honeywell UOP C₃ and C₄ Oleflex™ Processes, which produce polymer-grade propylene and iso-butene from propane and iso-butane feedstock, respectively, in a series of radial flow reactors. The Oleflex™ reactor section utilizes a highly selective, platinum-based catalyst system to dehydrogenate the light paraffin hydrocarbons. An example of an acceptable catalyst for light paraffin dehydrogenation is disclosed in U.S. Pat. No. 6,756,340, herein incorporated by reference. The reaction zone includes multiple reactors and interstage heaters. Cooling and separation of the reactor effluent into a hydrocarbon-rich fraction and a hydrogen-rich vapor fraction, part of which is non-recycled net off gas, is provided in the Oleflex™ separation zone. The Oleflex™ separation process typically includes a reactor effluent compressor (“REC”), and a series of expanders and separation vessels commonly referred to as a cold box. The Oleflex™ Process is described in Chapter 5.1 of the Handbook of Petroleum Refining Processes, Third Ed. 2003, p. 5.3-5.10.

One example of a known catalytic heavy paraffin dehydrogenation process is the Honeywell UOP Pacol™ Process, which can be applied to the dehydrogenation of heavy paraffins in the C₆-C₂₀ range. In the Pacol™ process linear paraffins are dehydrogenated to linear olefins in the presence of hydrogen over a selective platinum dehydrogenation catalyst. An adiabatic radial-flow reactor with feed preheat is conventionally utilized to compensate for the endothermic temperature drop and to minimize pressure drop within an efficient reactor volume. Hydrogen and some by-product light ends are separated from the dehydrogenation reactor effluent, and part of this hydrogen gas is recycled back to the dehydrogenation reactor. The Pacol™ Process is described in Chapter 5.2 of the Handbook of Petroleum Refining Processes, Third Ed. 2003, p. 5.11-5.19.

Other commercial processes are known for light and heavy paraffin dehydrogenation. However, there remains a need for improved equilibrium hydrocarbon dehydrogenation conversion, selectivity, and yield per pass.

### SUMMARY OF THE INVENTION

A process for dehydrogenation of a hydrocarbon feed includes providing the hydrocarbon feed in a feed stream to an inlet of a reactor. The hydrocarbon feed includes at least one hydrocarbon selected from light paraffins, heavy paraffins, or combinations thereof. The process further includes introducing an inert diluent into the feed stream, contacting the feed stream and the inert diluent with a catalyst in the reactor under dehydrogenation reaction conditions, and removing an effluent stream from the reactor at an outlet.

A process for dehydrogenating a light paraffin feed includes providing the light paraffin feed to an inlet of a reactor, where the light paraffin feed includes hydrogen and at least one of propane, butane, and pentane. The process further includes introducing at least one inert diluent selected from methane and nitrogen into the light paraffin feed, contacting the light paraffin feed and the inert diluent with a catalyst in the reactor under dehydrogenation reaction conditions, and removing an effluent stream from the reactor at an outlet. The light paraffin feed can have a ratio of hydrogen to light paraffin hydrocarbon in a range of about 0.1:1 to about 1.0:1 on a molar basis and a ratio of inert diluent to hydrocarbon in a range of about 0.1:1 to about 3.0:1 on a mole basis.

A process for dehydrogenating a heavy paraffin feed includes providing the heavy paraffin feed to an inlet of a reactor, where the heavy paraffin feed includes hydrogen and at least one C₆-C₂₀ paraffin. The process further includes introducing at least one inert diluent selected from methane and nitrogen into the heavy paraffin feed, contacting the heavy paraffin feed and the inert diluent with a catalyst in the reactor under dehydrogenation reaction conditions, and removing an effluent stream from the reactor at an outlet. The ratio of hydrogen to heavy paraffin hydrocarbon can be in a range of about 0.1:1 to about 10:1 on a molar basis and the ratio of inert diluent to hydrocarbon can be in a range of about 0.1:1 to about 3.0:1 on a mole basis.

### BRIEF DESCRIPTION OF THE DRAWINGS

FIG. 1 is a schematic diagram of a hydrocarbon dehydrogenation process;

FIG. 2 is a schematic diagram of an alternative configuration for the reactor of FIG. 1.

### DETAILED DESCRIPTION OF THE INVENTION

This invention relates to a dehydrogenation process for converting a hydrocarbon feed to mono- or di-olefin products. More specifically, the present process is directed to improving conversion and/or selectivity of a hydrocarbon dehydrogenation reaction by introducing inert diluent into a reactor. It has been discovered that in some cases conversion may be improved while maintaining or even decreasing the temperature drop across the reactor. Implementation of the present process in commercially available dehydrogenation processes advantageously may not require significant upgrades to existing systems. A minor upgrade to allow for introduction of the inert diluent might be expected, but replacing portions of the process equipment to accommodate different volumes or different materials such as water, for example, is not expected to be necessary. The present process can also be used in newly constructed dehydrogenation systems.

The present process provides an improved yield per pass of olefin (mono- or di-olefin) in a hydrocarbon dehydrogenation process by including the inert diluent with the hydrocarbon feed and contacting the diluent and feed with a catalyst in the dehydrogenation reactor. The reactor may be a reactor zone that includes multi-stages or multiple reactors, often in series. Some commercially available systems currently utilize three reactors in series to dehydrogenate isobutene and four reactors in series to dehydrogenate propane. Typically, one reactor, optionally a radial bed type, is utilized to dehydrogenate heavy paraffins.

Methane is utilized as an exemplary diluent in the present process. Other inert gases, such as nitrogen, helium and argon are also contemplated as acceptable inert diluents for the present process. Methane, nitrogen and other diluents advantageously limit the undesirable effects of employing steam as a diluent. By utilizing methane or nitrogen, for example, instead of steam, there is reduced or greatly reduced potential for undesirable side reactions, such as CO, CO₂, or oxygenate formation. The inert diluent will not strip chloride from the catalyst when a chloride containing dehydrogenation catalyst is employed. Steam diluent promotes corrosive by-products, which may require the metallurgy of process equipment to be upgraded, these upgrades are typically costly to accommodate. This effect is reduced when a diluent other than steam is employed. Additional advantages of the present inert diluent over steam include expected cost savings by reducing the use of energy intensive condensation and revaporization of the steam/water to recycle the diluent. While steam can be condensed and removed from the product stream 24 to a large extent, driers are required to remove water completely for light olefin recovery, which often requires extreme (substantially less than 0° C.) conditions. Further, the present process requires minimal changes to conventional processes which are already designed to remove non-condensable gases, such as hydrogen and methane, from desired products and unconverted hydrocarbon feed. Many light paraffin dehydrogenation units also employ a hydrogen purification system, such as a pressure swing adsorption (“PSA”) unit, to recover high purity hydrogen. A PSA unit is one example of a hydrogen purification system, but any other suitable hydrogen purification system can be employed.

Inert diluent has been found to have two effects: decreasing the partial pressure of the dehydrogenation reaction products, and increasing the enthalpy of the circulating gases in the reactors. Both effects, individually and in combination, allow higher hydrocarbon conversion before the reaction reaches equilibrium. Additional features of the present process provide economic benefits related to minimizing the need to retrofit or redesign existing process equipment.

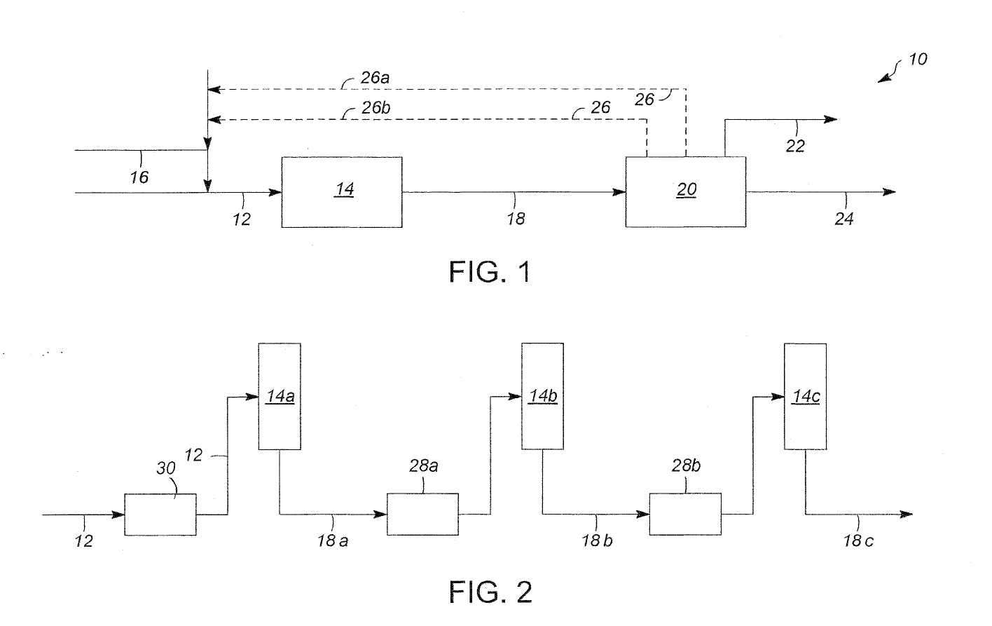

Various embodiments of the invention will now be discussed with respect to FIGS. 1-2. The drawings are schematic representations, which will be understood by artisans in view of the general knowledge in the art and the description that follows. Features may be exaggerated in the drawings for emphasis, and features may not be shown to scale.

Referring to FIG. 1, a process for dehydrogenation of a hydrocarbon feed is shown generally 10. The process includes providing the hydrocarbon feed in a feed stream 12 to an inlet of a reactor 14.

Contained in the feed is at least one hydrocarbon selected from light paraffins or heavy paraffins. The present process further provides introducing an inert diluent into the feed stream 12. Methane is an example of an inert diluent presently utilized; however other inert gases such as nitrogen, helium, argon, or a combination may also be acceptable. Next, the process provides contacting the feed stream 12 and the inert diluent with a catalyst in the reactor under dehydrogenation reaction conditions. The inert diluent shifts the equilibrium of the hydrocarbon dehydrogenation reaction toward production of more mono- or di-olefins due to reduction of the partial pressure of hydrogen and other products in the reactor 14, while simultaneously adding a heat carrier to the system. In some embodiments, the presence of the inert diluent, acting as a heat carrier, surprisingly results in the change in temperature (ΔT) across the reactor 14 or reactor zone decreasing or remaining constant while achieving an improved conversion. Thus, in such embodiments, the present process can provide dehydrogenation of a hydrocarbon feed wherein a temperature difference across the reactor is about the same as or less than a temperature difference across the reactor without the inert diluent, and wherein a conversion of hydrocarbon is increased compared to a conversion of hydrocarbon without the inert diluent.

The enthalpic effect of the inert diluent relative to the partial pressure reduction will vary depending on hydrogen to feed hydrocarbon ratio, diluent to hydrocarbon ratio, and feed hydrocarbon carbon number or carbon number range, among other things. In all cases, the presence of the inert diluent will increase the heat capacity (per mole of reactive hydrocarbon) of the stream directed to the reactor inlet at a given temperature. For example, at about 625° C., a stream containing propane, methane, and hydrogen with a methane to propane molar ratio of about 1.0 and a hydrogen to propane molar ratio of about 0.6 will have a heat capacity per mole of propane approximately 20% higher than a stream containing only hydrogen and propane in the same portions. A similar stream at about 625° C. containing isobutane (iC₄), hydrogen (H₂), and methane (C₁) with H₂/iC₄ molar ratio of 0.8 and C₁/iC₄ molar ratio of about 0.8 will have a heat capacity per mole of isobtane of approximately 12% higher than a stream containing only hydrogen and isobutane. As shown in the examples below, this increase in heat capacity per mole of reactive hydrocarbon in the feed stream will impact the temperature change in an adiabatic reactor. As shown in the examples below, this increase in heat capacity is expected to positively impact the temperature change in an adiabatic reactor.

Following the reaction, the present process provides for removing an effluent stream 18 from the reactor 14 at an outlet where the effluent stream includes at least one of a mono-olefin, di-olefin or a combination. Optionally, the reactor has an absolute outlet pressure within a range of about 0 to about 350 kPa. All pressures herein refer to absolute pressure unless stated otherwise. Light paraffin dehydrogenation will typically be carried out at a lower pressure than heavy paraffin dehydrogenation, as will be described below.

In some cases, the reactor is operated at or above atmospheric pressure to avoid the need for a vacuum system. For example, the reactor may be operated at a pressure of at least approximately 101 kPa or at least approximately 120 kPa. Depending on the feed, the effluent stream 18 includes at least one of a mono-olefin or a di-olefin. Many dehydrogenation processes cool the effluent stream before it is sent to a separation zone 20, which may include adsorbent beds such as a PSA unit, a membrane, a cold box, one or more heat exchangers or coolers, one or more expanders, one or more separators, or any combination of separation equipment as is known in the art. The separation zone 20 may optionally comprise a first separation system where a vapor phase of the effluent stream 18 is separated from a liquid phase, and a second separation system, where the inert diluent and hydrogen are separated. Inert diluents are typically in the non-condensable gas phase, while the hydrocarbon product is typically in the liquid phase.

Typically, the separation zone 20 includes one or more separator vessels in which vapor components are separated from heavier, condensed components. This separation zone 20 also includes equipment to achieve the desired temperatures and pressures for the desired phase separation. One skilled in the art will understand that the conditions will vary with the carbon number of the feedstock. In some instances, such as heavy paraffin dehydrogenation, the separation zone 20 may contain only a cooler and a simple separation vessel. In other applications, such as light paraffin dehydrogenation, separation may be more complex, and the separation zone 20 may include one or more of a heat exchanger, air cooler, reactor effluent compressor (“REC”), expander, turbo expander and multiple separation vessels. The additional equipment is used in light paraffin dehydrogenation in order to separate C₂+hydrocarbons from the methane and hydrogen that is recycled in the present process.

A second separator system (not shown) in the separation zone 20 may also be included for separating the inert diluents from the net hydrogen. This second separator system may include, but is not limited to, the use of membranes or adsorbents and will also include equipment required to achieve conditions, such as temperature and pressure, suitable for the desired separation. The second separator system may be located either be upstream or downstream of the first separation system in the separation zone 20. An advantage of including the second separation system is that it enables the recovery of the inert diluent from the hydrogen and other light byproducts, such as methane, produced in the process. Thus, the present process provides for separation of the inert diluent from hydrogen so each can be circulated back to the reactor 14 in the desired portions. Inclusion of the second separator in the present process reduces consumption of inert diluent on a continuous basis via stream 16 by enabling recycle of the inert diluent to the reactor. Thus, the second separation system allows a reduction in the addition of inert diluent via stream 16. Therefore, a cost savings is realized due to the reduction of new or make-up diluent that must be supplied.

The relative positions of the first and second separation systems will vary, depending on the number of carbons in the hydrocarbon feed 12 and the technologies employed for the separation. In one embodiment, discussed below, the present process is applied to light hydrocarbon dehydrogenation and utilizes a hydrogen selective membrane in the second separation zone, and it may be advantageous to locate the second separator upstream of the first separator. In another embodiment, the second separation zone employs a hydrogen selective membrane or adsorbent technology such as pressure swing adsorption. In this embodiment the second separation system can be advantageously located downstream of the first separation system in the separation zone.

Hydrogen rich net gas 22 is separated from product stream 24 and recycle streams 26a and 26b in the present process. A vapor fraction of the effluent 18 (FIG. 1) containing predominantly hydrogen can be separated and then divided into recycled 26a and non-recycled 22 portions. The non-recycled portion 22 is a net separator off gas containing the net hydrogen produced in the catalytic dehydrogenation process. In some cases the net separator off gas may also include light byproducts such as but not limited to methane The recycled portion 26_{la,b} of the vapor fraction is conventionally combined with the hydrocarbon feed 12 to the catalytic dehydrogenation process reactor 14.

The processes of the present invention are useful for the dehydrogenation of hydrocarbons. Hydrocarbon dehydrogenation processes which could advantageously employ the present process of improving conversion by introducing an inert diluent into the feed include light paraffin dehydrogenation to mono- or di-olefins and heavy paraffin dehydrogenation to mono- or di-olefins.

Turning to FIG. 2, a reactor, as described above, may include a “reactor zone” having multiple reactors 14_{a-c} in series, often referred to as a multistage reactor system. The reactor zone has a reactor zone inlet corresponding to an inlet of a first reactor 14ₐ in the reactor zone. In one embodiment, the reactor zone 14 may include one or more adiabatic radial flow reactors. In another embodiment, the reactor zone 14 may include one or more adiabatic radial flow reactors that circulate catalyst to a regeneration zone (not shown) continuously or semi-continuously as is commonly practiced in light paraffin dehydrogenation. In yet another embodiment, the reaction zone 14 may include one or more fixed bed radial flow reactors in series or in parallel with one or more of the reactors in operation at any given time. The reactor zone 14 may also include equipment for heating the feed 12, such as a fired heater 30, to a desired inlet temperature.

The reactor outlet providing the effluent stream 18 will be the outlet of the last reactor 14_{c} in the reactor zone. Interstage reheating 28 is utilized between the multiple reactors 14_{a-c} in the reactor zone to increase the equilibrium conversion level because, as is known in the art, hydrocarbon dehydrogenation is an endothermic reaction. Thus, adding heat between the reactors 14_{a-c} in the reactor zone favors the desired dehydrogenation products. Heat addition is commonly achieved by a combination of heat exchangers and fired heaters. Heat may also be provided, at least in part, by heat stored in the catalyst that is present in the reactors.

Light paraffin dehydrogenation is an example of an advantageous embodiment of the present process. In this embodiment, at least one hydrocarbon in the feed stream 12 is a light paraffin. The feed stream further comprises hydrogen and the light paraffin, which can include at least one of propane, butane, and pentane. Propylene, a dehydrogenation product of propane, has significant commercial value. The feed stream 12 can have a ratio of hydrogen to the light paraffin in a range of about 0.1:1 to about 1.0:1 on a molar basis, optionally a ratio of about 0.1:1 to about 0.8:1, or optionally about 0.3:1 to about 0.7:1. A ratio of inert diluent to light paraffin can be in a range of about 0.1:1 to about 3.0:1 on a molar basis. Optional ranges include about 0.2:1 to about 2.0:1 and about 0.5:1 to about 1.5:1.

As discussed above, dehydrogenation of paraffins to produce olefins is a reversible endothermic reaction which is limited by equilibrium at the reactor outlet conditions. In order to increase conversion of the paraffinic feed, the reaction conditions must be manipulated to favor olefin production by increasing the temperature or reducing the partial pressure of hydrogen and the olefin product. While raising the temperature causes problems associated with thermal cracking side reactions and increased rates of coking, lowering the pressure presents another set of problems. In the case of light paraffin dehydrogenation, substantially decreasing the reactor pressure would require operating the reactor effluent compressor suction under vacuum. While dehydrogenation of heavier paraffins typically is not practiced under vacuum, lower pressure operation in this case does suffer from disadvantages of larger equipment size and utilities.

One commercially available propane dehydrogenation process is the Honeywell UOP Oleflex™ C₃ Process, which is described in U.S. Pat. No. 3,978,150, herein incorporated by reference. A propane-containing gas feed stream 12 is typically preheated to a temperature usually in the range of about 550° C. to about 700° C., optionally about 600° C. to about 675° C. Dehydrogenation occurs in the multi-stage reaction zone having four radial flow platinum-based catalytic reactors and producing effluent 18 that is normally a gas stream containing predominantly unreacted propane, propylene, hydrogen, and some non-selective reaction products (or byproducts). Heating the effluent stream 18_{a-c} from one reactor before it enters the next reactor is optionally provided in this process embodiment. As described above, the reactor zone has inlet corresponds to the inlet of the first reactor in the reactor zone and the reactor zone outlet similarly corresponds to the outlet of the last reactor in the zone.

As described above, light paraffin dehydrogenation typically takes place at lower pressure than heavy paraffin dehydrogenation. Thus, in this embodiment, the reactor outlet pressure can be within a specified range of about 0-175 kPa, or optionally about 101-175 kPa, or about 120-175 kPa.

Introducing an inert diluent to the feed 12 and circulating it in the reactor 14 (or the reactor zone) decreases the partial pressure of the key reaction products, hydrogen and propylene at the reactor outlet and maintains a higher temperature in the reactor 14 at a given conversion, allowing the reactor 14 to operate more isothermally. This may be referred to as the “enthalpic effect” of the inert diluent in the present process. Circulating inert diluent in the reactor 14 enables higher conversion to be achieved. For example, without the inert diluent present, operation at sub-atmospheric pressure may be required to reach a desired conversion level, where as the inert diluent may allow operation of the reactor 14 and separation zone 20 at or above atmospheric pressure. Thus, an economic advantage is expected by avoiding operation under vacuum conditions (sub-atmospheric pressure), which may be significantly more costly. Surprisingly, addition of an inert diluent in the present process provides a significant gain in conversion of propane to propylene even if the reactor 14 outlet pressure is maintained or increased compared to that of a dehydrogenation process without inert diluent added. Thus, operation at vacuum can advantageously be avoided, while realizing an improved product yield.

The yield per pass of propylene, for example, at a constant or decreased ΔT, can be increased over that which is obtainable in the same propane dehydrogenation process without the inert diluent. A yield per pass of propylene, for example, provided by the present process is at least about 30%, preferably at least about 32% and most preferably at least about 35%. This surprising result is believed to be due to the synergy of decreasing the partial pressure to shift the equilibrium amount of product, combined with the inert diluent acting as a heat carrier. If the improvement was merely equilibrium based, a greater quantity of diluent would be required than is needed to achieve the present results.

In one embodiment of the present process for light paraffin dehydrogenation, methane is added to a propane feed at a molar ratio of methane to propane of approximately 1.1:1.0, while maintaining the reactor zone outlet pressure and hydrogen to propane ratio of the feed. Under these conditions, the conversion per pass is increased from a base case by about 10%. Simultaneously, the catalyst selectivity is increased by about 1.5% (by weight). As is known in the art, conversion, yield-per-pass and selectivity are proportionately related.

Light paraffin dehydrogenation processes generally require more complex separation steps, as described above. Circulating inert diluent may have the practical effect of increasing the molar flow to the REC by approximately 30%, which will have an impact on the cost of operating the compressor. Therefore, the REC suction pressure may be increased proportionally to maintain an actual volumetric flow at the REC inlet that is equivalent to the actual volumetric flow at the REC inlet before the inert diluent (methane) is circulated. This advantageously allows an improved conversion and yield per pass without the necessity of changing the existing processing equipment, such as reactors, compressors and pipelines connecting the various pieces of equipment in the process, which would be expected in order to accommodate a greater volume of gases through the reactor section.

Advantageously, the present process enables the compressor, which is one of the largest pieces of equipment in the dehydrogenation system, as well as the conduits carrying gases, to be kept the same size. Thus, it was surprisingly discovered that the present process provides a significant increase in output, but does not require an investment in resized or new equipment. Even with elevated reactor outlet pressure, the present process provides improved performance in terms of conversion, selectivity, and yield per pass. The improved yield-per-pass, conversion and selectivity performance of the present process is believed to be attributable to the enthalpic effects of the inert diluent, sometimes manifested as a decreased ΔT across the reactor zone, in combination with the decreased partial pressures of hydrogen and other reaction products, such as propylene, at the reactor zone outlet. In the case of a propane feed, a purified propane fraction is conventionally obtained from a propylene recovery unit (PRU), which is then dehydrogenated over the catalyst in the reactor zone along with fresh propane feed. Actual conversion, selectivity, and yield per pass depend on the composition of the hydrocarbon feed and the operating conditions. A hydrocarbon with a lower carbon number has a lower heat capacity than a hydrocarbon feed with a higher carbon number. Heat capacity is defined as an amount of energy required to raise the temperature of a material, for example the energy required to increase the temperature of one mole by 1° C.

In another embodiment of the present process, dehydrogenation of a hydrocarbon is provided wherein at least one hydrocarbon is a heavy paraffin. The feed in this embodiment includes hydrogen and the hydrocarbon includes at least one C₆-C₂₀ paraffin.

One commercially available heavy paraffin dehydrogenation process is the Honeywell UOP Pacol™ Process. A C₆-C₂₀ paraffin feed 12 stream is typically preheated to a temperature in the range of about 400° C.-550° C., and optionally 430-500° C. Heavy hydrocarbon dehydrogenation typically occurs in a radial bed reactor, producing effluent 18 (FIG. 1) containing predominantly olefins, hydrogen, and the non-selective reaction products (or byproducts).

An embodiment of the present process for dehydrogenating a heavy paraffin feed includes providing the heavy paraffin feed to the inlet of the reactor 14. The heavy paraffin feed includes hydrogen and at least one C₆-C ₂₀ paraffin. At least one inert diluent, such as methane, nitrogen, helium and argon, is introduced into the heavy paraffin feed before contacting the feed and diluent with a catalyst in the reactor under dehydrogenation conditions and removing an effluent stream from the reactor at an outlet. The heavy paraffin feed of this embodiment has a ratio of hydrogen to heavy paraffin in a range of about 0.1:1 to about 10:1. Optionally, this ratio is about 2:1 to about 10:1, or from about 3:1 to about 7:1. Inert diluent is present in a ratio of inert diluent to paraffin in a range of from about 0.1:1 to about 3.0:1, or from about 0.2:1 to about 2.0:1, or from about 0.5:1 to about 1.5:1. As discussed above, heavy paraffins are typically dehydrogenated at higher pressures than light paraffins. In the present embodiment, the reactor outlet pressure is in a range of 0 to about 350 kPa, optionally about 101 to about 350 kPa, or about 120 kPa to about 350 kPa.

The following examples are presented for the purpose of illustration only and are not intended to limit the scope of the present invention. The examples illustrate the significance of an inert diluent in achieving an increase in conversion of a hydrocarbon dehydrogenation reaction.

TABLE 1

| | | | Add Inert |
|---|---|---|---|
| | Base | Add Inert | Diluent and |
| | Case | Diluent | Increase Pressure |
| C₁/C₃ at R1 Inlet, | 0.0 | 1.1 | 1.0 |
| mol/mol | | | |
| H₂/C₃ R1 Inlet, | 0.6 | 0.6 | 0.6 |
| mol/mol | | | |
| R4 Outlet Pres, kPa | 136 | 136 | 170 |
| R4 Effluent, | 9,894 | 11,915 | 12,284 |
| kmol/hr | | | |
| Conversion, % | Base | Base + 10.1 | Base + 6.9 |
| Selectivity, wt % | Base | Base + 1.6 | Base + 0.1 |
| Yield per pass, wt % | Base | Base + 9.3 | Base + 6.3 |
| Total ΔT (° C.) | Base | Base − 2 | Base − 14 |

Referring to Table 1, the benefit of circulating methane in the reactor section of an Oleflex™ C₃ propane dehydrogenation system was modeled using a process simulator and reactor model. Column 1 depicts the base case, assuming a hydrogen to propane (H₂/C₃) reactor inlet molar ratio of 0.6. Also assumed was a 136 kPa outlet pressure of a fourth reactor in series (R4), which was the last reactor in the reactor zone.

By adding methane and keeping the R4 outlet pressure at 136 kPa, the conversion of propane to propylene increased from a base by approximately 10 wt % without changing the reactor inlet temperatures, while the temperature drop across the reactors surprising remained the same or decreased.

As shown in column 3 of Table 1, methane was added and the R4 outlet pressure was increased. While the conversion rate was improved over the base case by about 7 wt %, the R4 effluent molar flow rate increased 34% from 9,148 kmol/hr to 12,284 kmol/hr. This increase was largely attributable to the addition of methane. In a desirable embodiment, this increased molar flow rate is accommodated without increasing the size of the Oleflex™ process equipment by increasing the REC suction pressure proportionately to the increase in effluent flow rate from R4 to the REC. Assuming the REC suction drum operates at a minimum pressure of about 101 kPa, atmospheric pressure, the system pressure must be increased by about 34 kPa to compensate for the increased molar flow to the compressor. Thus, the original process equipment size can be maintained, while still realizing an improved conversion, yield per pass, and selectivity.

TABLE 2

| | Base | Add | Add Methane |
|---|---|---|---|
| | Case | Methane | Increase Pres |
| C1/iC4 at R1 inlet, | 0.1 | 0.8 | 0.8 |
| mol/mol | | | |
| H2/iC4 R1 Inlet, | 0.8 | 0.8 | 0.8 |
| mol/mol | | | |
| R4 Outlet Pres, kPa | 134 | 134 | 168 |
| R4 Effluent, kmol/hr | 4569 | 6020 | 5963 |
| Conversion, % | Base | Base + 4.6 | Base + 1.8 |
| Selectivity, wt % | Base | Base + 0.5 | Base + 0.1 |
| Yield per pass, wt % | Base | Base + 4.4 | Base + 1.7 |
| Total Delta T, ° C. | Base | Base + 22 | Base + 9 |

Referring to Table 2, the benefit of circulating methane in the reactor section of an Oleflex™ C₄ isobutane dehydrogenation system was modeled.

By adding methane and keeping the reactor outlet pressure unchanged, the conversion of isobutane to isobutene increased by about 4%. In this case, the temperature change increased, but the conversion, selectivity and yield per pass still showed improvement. This increase in ΔT is believed to be consistent with the previously described enthalpic effect of the inert diluent as the heat capacity of propane is lower than that of isobutane. Therefore, the relative impact of methane as a heat carrier is larger for propane. Also, the impact on temperature change across the reactor for a heavy paraffin feed is expected to be smaller than for light paraffin because the heavy paraffin reaction mixture commonly includes higher hydrogen to hydrocarbon ratios.

As shown in column 3 of Table 2, methane was added and the reactor/reactor zone outlet pressure was increased. While the conversion rate was improved over the base by about 1.8%, the reactor effluent flow rate increased, and is attributable to methane, as in Example 1. This increased molar flow rate is also accommodated as described above.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims. Various features of the invention are set forth in the appended claims

What is claimed is:

1. A process for dehydrogenation of a hydrocarbon feed, comprising:
providing the hydrocarbon feed in a feed stream to an inlet of a reactor, the hydrocarbon feed comprising at least one hydrocarbon selected from light paraffins or heavy paraffins;
introducing an inert diluent into the feed stream;
contacting the feed stream and the inert diluent with a catalyst in the reactor under dehydrogenation reaction conditions; and
removing an effluent stream from the reactor at an outlet.

2. The process of claim 1 wherein said effluent stream comprises at least one of a mono-olefin and a di-olefin.

3. The process of claim 1 wherein the reactor outlet has an absolute pressure in a range of 0 to about 350 kPa.

4. The process of claim 1, wherein the inert diluent is methane, nitrogen, helium, argon, or a combination thereof.

5. The process of claim 1, wherein the at least one hydrocarbon is the light paraffins, wherein the feed stream further comprises hydrogen, and wherein the light paraffins comprise at least one of propane, butane and pentane, the feed stream having a ratio of hydrogen to the light paraffins in a range of about 0.1:1 to about 1.0:1 on a molar basis.

6. The process of claim 5 wherein a ratio of inert diluent to light paraffin is in a range of about 0.1:1 to about 3.0:1 on a molar basis.

7. The process of claim 1, wherein the at least one hydrocarbon is the heavy paraffins, wherein the feed stream further comprises hydrogen, and wherein the heavy paraffins comprise at least one C₆-C₂₀ paraffin, the feed stream having a ratio of hydrogen to the heavy paraffins in a range of about 0.1:1 to about 10:1 on a molar basis.

8. The process of claim 1 wherein the reactor is operated at or above atmospheric pressure.

9. The process of claim 1 wherein the reactor comprises a reactor zone having at least two reactors in series, the reactor zone having a reactor zone inlet corresponding to an inlet of a first reactor in the reactor zone and a reactor zone outlet corresponding to an outlet of a last reactor in the reactor zone.

10. The process of claim 9 further comprising heating an effluent stream from one reactor before it enters the next reactor.

11. The process of claim 1 further comprising heating the feed stream.

12. The process of claim 1 wherein a yield of hydrocarbon per pass is increased compared to a yield of hydrocarbon per pass without the inert diluent.

13. The process of claim 1 further comprising separating at least one vapor phase and at least one liquid phase from the effluent stream in a separation zone.

14. The process of claim 13 further comprising separating a portion of the inert diluent from the vapor stream for recycling to the feed stream.

15. The process of claim 13 further comprising separating a portion of hydrogen from the vapor phase for recycling to the feed stream.

16. The process of claim 13 wherein the separation zone includes a first separation system and a second separation system.

17. A process for dehydrogenating a light paraffin feed, comprising:
providing the light paraffin feed to an inlet of a reactor, the light paraffin feed comprising hydrogen and at least one light paraffin of propane, butane or pentane;
introducing at least one inert diluent selected from methane and nitrogen into the light paraffin feed;
contacting the light paraffin feed and the inert diluent with a catalyst in the reactor under dehydrogenation reaction conditions; and
removing an effluent stream from the reactor at an outlet;
wherein the light paraffin feed has a ratio of hydrogen to light paraffin in a range of 0.1:1 to 1.0:1 on a molar basis and a ratio of inert diluent to light paraffin in a range of about 0.1:1 to about 3.0:1 on a molar basis.

18. The process of claim 17 wherein the reactor outlet pressure is in a range of 0 to about 175 kPa.

19. A process for dehydrogenating a heavy paraffin feed, comprising:
providing the heavy paraffin feed to an inlet of a reactor, the heavy paraffin feed comprising hydrogen and at least one C₆-C₂₀ paraffin;
introducing at least one inert diluent selected from methane and nitrogen into the heavy paraffin feed;
contacting the heavy paraffin feed and the inert diluent with a catalyst in the reactor under dehydrogenation reaction conditions; and
removing an effluent stream from the reactor at an outlet;
wherein the heavy paraffin feed has a ratio of hydrogen to heavy paraffin in a range of 0.1:1 to 10:1 on a molar basis and a ratio of inert diluent to paraffin in a range of about 0.1:1 to about 3.0:1 on a mole basis.

20. The process of claim 19 wherein the reactor outlet pressure is in a range of 0 to about 350 kPa.
